(19) United States
(12) Patent Application Publication
(10) Pub. No.: US 2001/0014794 A1
(43) Pub. Date: Aug. 16, 2001

(54) Medical device

(76) Inventors: Franciscus Laurens Moll, Dublin (IE); Menno Kalmann, Dublin (IE)

Correspondence Address: JACOBSON, PRICE, HOLMAN & STERN, PROFESSIONAL LIMITED LIABILITY COMPANY, 400 SEVENTH STREET N.W., WASHINGTON, DC, 20004, US

(21) Appl. No.: 09/780,425

(22) Filed: Feb. 12, 2001

(30) Foreign Application Priority Data
Feb. 11, 2000 (NL) 1014364
Mar. 3, 2000 (NL) 1014559

(51) Int. Cl. A61M 35/00

(52) U.S. Cl. 604/289

(57) ABSTRACT

An endoluminal side branch graft comprises a generally tubular body having a proximal part with a proximal opening, a distal part with a distal opening, and a middle section extending between the proximal and distal parts, at least one of the proximal or distal parts has fixing means for fixedly positioning the side branch graft to a main branch graft, the fixing means having a delivery configuration and a stable fixing configuration. A primary graft is first introduced to a site in the body conduit having a side branch. The position of the side branch which has been closed off by the primary graft is mapped and an opening is provided in the primary graft at the mapped position of the side branch. A side graft is introduced into the side branch and fixed to the primary graft.

### INTRODUCTION

The invention relates to an endoluminal side branch graft, to a graft assembly incorporating such a side branch graft, and to the use of such grafts and graft assemblies.

An aneurysm is a swelling in an artery that is usually caused by localised damage or weakness of the vessel wall. Such aortic aneurysms are a significant medical problem and affect a significant proportion of the population. Treatment involves replacing or reinforcing the affected segment of the artery with a graft which is typically of a pliable polymeric material such as expanded polytetrafluroethylene, woven polyester or Teflon. Aneurysms may be treated using a surgical repair technique. However the less invasive technique of endovascular grazing is becoming more widely used. In this technique a prosthetic arterial graft is placed transluminally within the lumen of the affected artery. The graft is anchored in position in the vessel, typically by using a radially expandable stent.

The aorta defines a complex tortuous passageway which has a number of side branches. There are several such branch arteries including subclavian arteries carotid arteries, intercostal arteries, renal arteries, the superior mesenteric artery, and the inferior mesenteric artery. There are particular problems in deploying endovascular grafts in regions of the aorta from which such side branches extend because in many cases the flow of blood to the side branches must be maintained.

One technique to address this problem involves providing an endovascular graft with pre-formed openings for alignment with side branches. However, this technique is not satisfactory because a very wide range of such grafts must be made available to the clinician to cater for the various locations in the aorta at which side branches may occur. In addition, the tortuosity of the aorta varies widely from one patient to another and it is consequently very difficult to provide an appropriate graft to cater for the wide variations required.

This invention is therefore directed towards providing a side branch graft, a graft assembly and a process for using such grafts and graft assemblies which will address at least some of these problems.

### STATEMENTS OF INVENTION

According to the invention there is provided an endoluminal side branch graft comprising a generally tubular body having a proximal part with a proximal opening, a distal part with a distal openings and a middle section extending between the proximal and distal parts, at least one of the proximal or distal parts having fixing means for fixedly positioning the side branch graft to a main branch graft, the fixing means having a delivery configuration and a fixing configuration.

In one embodiment of the invention the delivery configuration is a stable configuration, the fixing configuration is a stable configuration, and the fixing means comprises an integral locking means to lock the fixing means in the stable fixing configuration.

In another embodiment the fixing means comprises a plurality of fingers which extend longitudinally in the delivery configuration and which extend substantially radially outwardly in the fixing configuration. Preferably the fingers are circumferentially spaced-apart in the fixing configuration. Ideally a webbing extends between the fingers.

In one case the locking means comprises an elbow hinge.

In another case the locking means comprises an elbow hinge between the fingers and the body of the stent graft.

In a preferred embodiment the graft comprises anchoring means on the tubular body of the graft. Preferably the anchoring means comprises one or more hook-like elements for anchoring the graft in position in a side branch.

Desirably the graft is of PTFE, polytetrafluoroethylene.

According to another aspect the invention provides an endoluminal side branch graft system comprising a endoluminal side branch graft and a sheath for delivery or retrieval of the graft.

In one embodiment the sheath is arrangeable over the side branch graft for introduction and/or removal of the graft from its use site, the sheath when arranged on the side branch graft retaining the fixing means in the delivery configuration.

Preferably the system comprises a guide for guiding the assembly to and from the side branch graft use site. Ideally the guide comprises a guidewire over which the system is guideable.

In a preferred embodiment the system comprises a catheter having an internal diameter substantially greater than the diameter of the side branch graft, whereby the side branch graft is insertable/removable from its use site via an access opening arranged in the mother catheter.

In a further embodiment the system comprises a stabiliser to maintain the fixing means in the fixing configuration. Ideally the stabiliser is a stent to urge the fixing means into the fixing configuration.

According to a further aspect the invention provides endoluminal graft assembly comprising a endoluminal side branch graft and/or an endoluminal side branch graft system and a main branch graft.

The invention also provides use of an endoluminal side branch graft and/or a endoluminal side branch system and/or an endoluminal graft assembly for comprising a fluid-flow within a catheter and a body vessel particularly a blood vessel, closed off on insertion of said catheter.

According to the invention there is further provided a method for endoluminal grafting of a body conduit at a site having a side branch comprising the steps of:

introducing a primary graft to a site in the body conduit having a side branch;

mapping the position of the side branch which has been closed off by the primary graft;

providing an opening in the primary graft at the mapped position of the side branch;

introducing a side graft into the side branch; and

fixing the side graft to the primary graft.

Preferably the position of the side branch is mapped by rotational angiography.

Ideally the opening in the primary graft is provided by in situ cutting of a side opening in the graft.

The primary graft may be in situ cut from the inside of the primary graft.

The primary graft may be in situ cut from outside through the side branch.

The opening is preferably provided by laser cutting.

The side graft may be introduced through the side branch.

The side graft may be introduced through the primary graft.

In a preferred embodiment the side graft comprises fixing means for fixing the side graft to the primary graft, the fixing means having a delivery configuration and a fixing configuration, and the method includes the step of fixing the side graft to the primary graft by moving the fixing means from the delivery configuration to the fixing configuration. Preferably the fixing means is moved by an actuator and the method comprises the step of introducing the actuator to the fixing means endoluminally. Ideally the actuator comprises an actuating balloon and the method comprises the step of inflating the balloon to move the fixing means from the delivery configuration to the fixing configuration.

In another preferred embodiment the method comprises the steps of:

introducing a stabiliser to the fixing means; and actuating the stabiliser to maintain the fixing means in the fixing configuration.

In another aspect the invention provides a method for endoluminal grafting of a body conduit at a site having a side branch comprising the steps of:

introducing a primary graft to a site in the body conduit having a side branch;

mapping the position of the side branch which has been closed off by the primary graft;

in situ cutting an opening in the primary graft at the mapped position of the side branch by introducing a cutter through the primary graft;

introducing a side graft through the primary graft, and into the side branch; and

through the opening in the primary graft fixing the side graft to the primary graft.

In a further aspect the invention provides a method for endoluminal grafting of a body conduit at a site having a side branch comprising the steps of:

introducing a primary graft to a site in the body conduit having a side branch;

mapping the position of the side branch which has been closed off by the primary graft;

in situ cutting an opening in the primary graft at the mapped position of the side branch by introducing a cutter through the side branch;

introducing a side graft through the side branch; and

fixing the side graft to the primary graft.

Utilising a device according to the present invention, the supply of blood can be kept open into blood vessels branching off from the aorta, which branched blood vessels have been closed off due to the insertion of a mother graft into the aorta to treat an aneurysm, for example.

The side branch technology of this invention may be applied in particular to a Juxta renal abdominal aortic aneurysm in which one or both of the renal arteries and/or the superior mesenteric artery and/or the inferior mesenteric artery and/or coeliac trunk are provided with side branch grafts according to the invention. This is an especially difficult aneurysm to treat intraluminally because access is only available through the main artery. It is not possible to gain access to the side branches working from the outside in as the side branches lead to body organs. Therefore, in this instance the/or each side branch graft is fitted by gaining access through the primary or mother graft.

The side branch technology of the invention may also be applied particularly in treating an aneurysm in which at least some of the side branches are accessible from the outside. Such side branches include the subclavian and carotid(?) arteries. In these cases a guidewire may be readily placed in the side branches prior to deployment of a primary graft to provide rapid access for forming an opening in the primary graft and for deploying a side branch graft.

The side branch technology can also be used to simplify the grafting procedure at furcations in the artery, particularly an abdominal aortic aneurysm below the renal arteries. The term side branch as used in this specification includes such furcations.

It will be appreciated that the side branch technology of the invention may be used for grafting any suitable aneurysm. Indeed, for certain aneurysms the outside-in technique may be used to graft one or more side branches and the inside-out technique may be used to graft other side branches.

The side branch connection can be made very quickly using the device and techniques of the invention. This is particularly important in order to maintain oxygenated blood supply to vital organs.

### BRIEF DESCRIPTION OF THE DRAWINGS

The invention will be more clearly understood from the following description thereof given by way of example only with reference to the accompanying drawings in which:

FIG. 1 shows a stent in an unfolded position;

FIG. 2 shows a partially cut away perspective view of a part of the assembly according to the present invention;

FIG. 3 shows a partially cut away perspective view of a device according to the present invention, wherein the fixing positioning means are opened;

FIG. 4 shows a second embodiment of a device according to the present invention;

FIG. 5 shows a second preferred embodiment of a device according to the present invention;

FIGS. 6 to 13 show successively partially cut away views of insertion of the device according to the present invention into a blood vessel branching off from the aorta, which has suffered an aneurysm;

FIG. 14 shows a partially cut away perspective view of a second manner of insertion of the assembly according to the present invention.

FIGS. 15 and 16 show partially cut away side views of a further preferred embodiment of the device according to the present invention.

FIGS. 17 to 22 are perspective, partially cut-away views of steps in a side branch grafting technique in which a side branch graft is placed in a side branch by access through a main graft;

FIGS. 23 to 28 are perspective, partially cut-away views of steps in a side branch grafting technique in which a side branch graft is placed in a side branch by access through the side branch; and

FIGS. 29 to 31 are perspective, partially cut-away views of steps in another application of the side branch grafting technique in which a side branch graft is placed in a side branch by access through the side branch.

### DETAILED DESCRIPTION

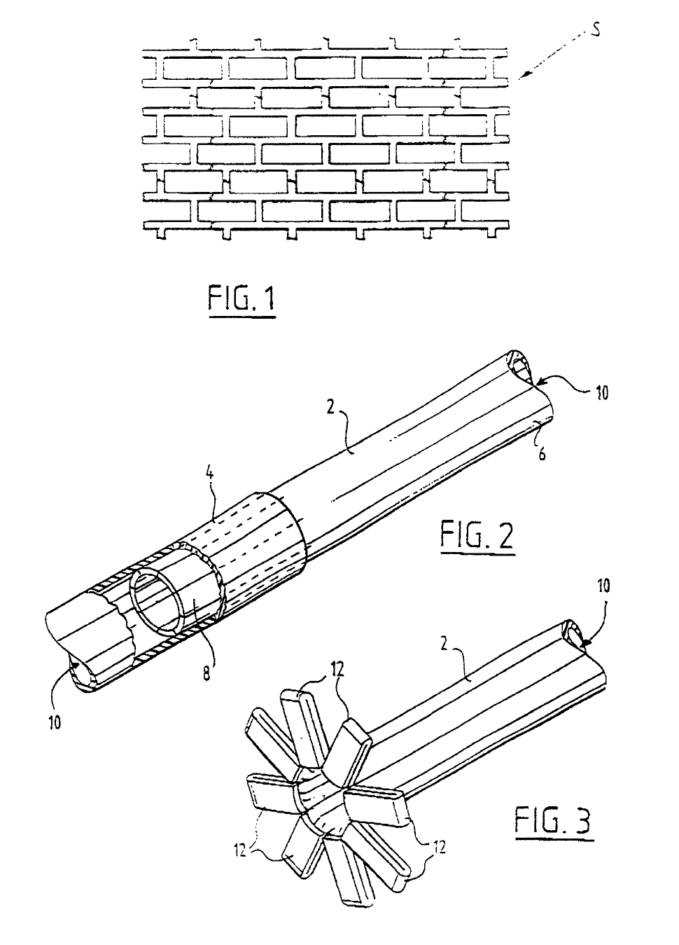

The device according to the present invention comprises a flat stent section S (see FIG. 1), which may be rolled up and covered with a graft to provide a generally cylindrical side branch graft device 2 (see FIG. 2).

An assembly according to the present invention comprises the cylindrical connecting graft device 2 and a sheath 4 arranged thereover.

The graft device 2 has a distal part 6, a proximal part 8 and an internal fluid channel 10 extending through the graft device 2.

The proximal part 8 is provided with eight separate finger sections 12, which are pretensioned to assume an open, umbrella-like position (see FIG. 3), when the sheath 4 is removed.

When the sheath 4 is arranged over the graft device 2, the fingers 12 are however closed, so that the fingers 12 are continuous with the body of the graft device 2, to provide a uniform cylindrical form in the delivery configuration.

The graft device 2 is preferably made of thin biocompatible graft material such as PTFE.

The graft device 2 can also be provided with hingable hook-like elements 14 arranged therealong to provide an extra anchoring force, when the device is arranged in position (see FIG. 4).

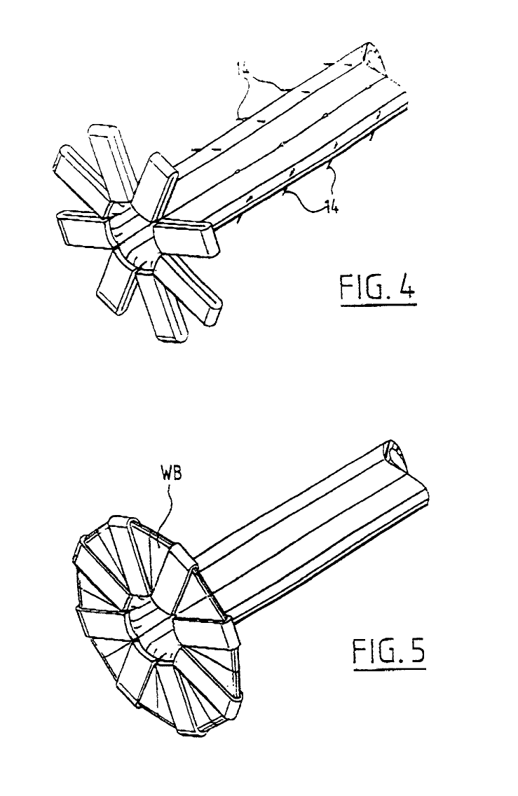

In a second preferred embodiment of the graft device (FIG. 5), material webbing WB is arranged between the finger sections 12. This webbing W can be made of any suitable material and further aids in anchoring the device in position and prevents unwanted matter from accumulating between the fingers.

The device is used as follows:

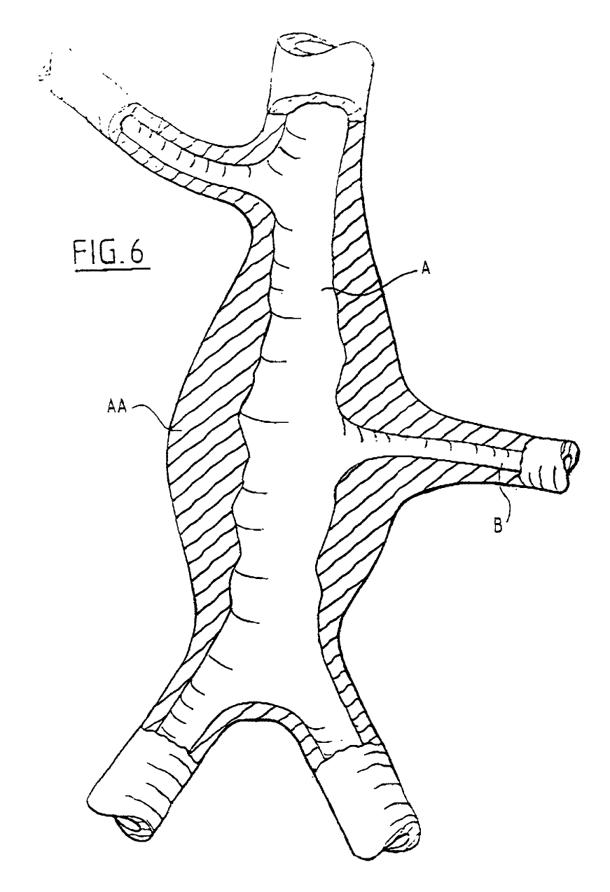

The treatment of an aortic aneurysm is often carried out by inserting a balloon catheter into the aorta to occlude the aortic aneurysm without interfering with cerebral, coronary or renal blood supply. An aneurysm, a swelling in the aorta A, is shown in FIG. 6, designated AA.

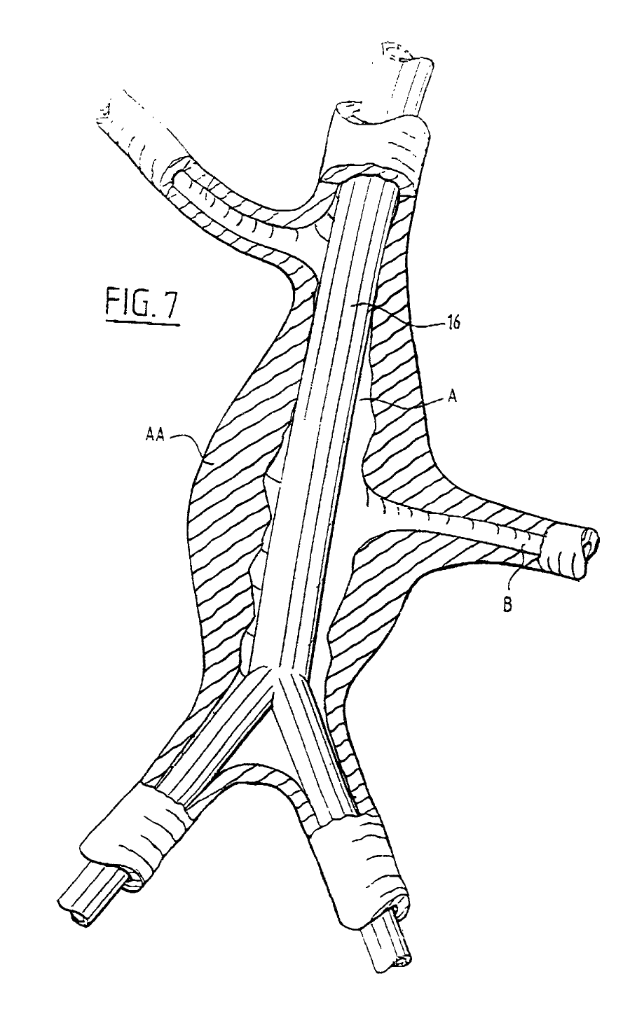

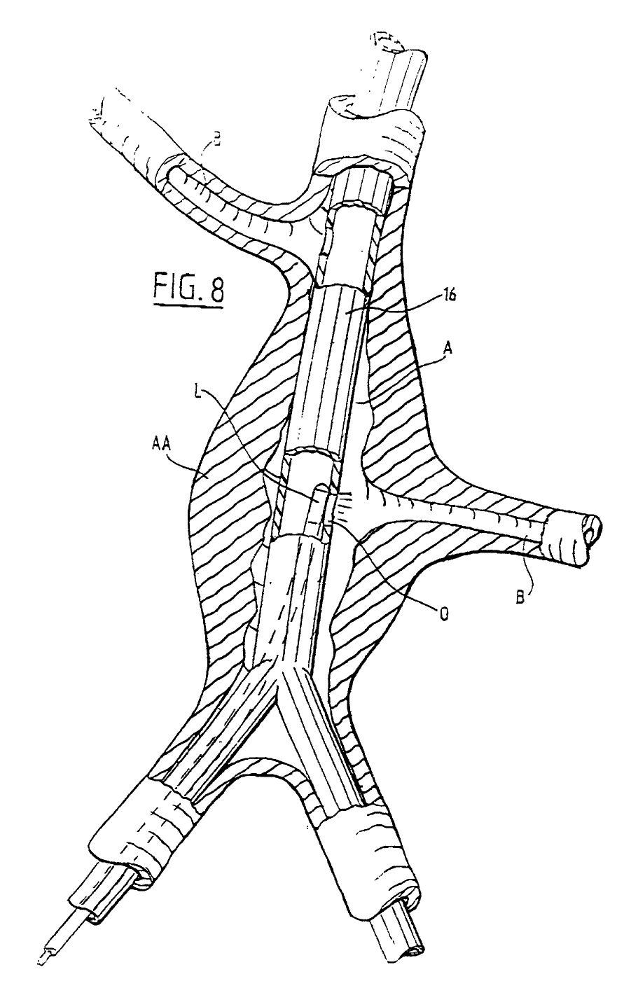

A mother or primary graft 16 is inserted via the groin into the aorta A to occlude the aneurysm (FIG. 7). On introduction of this mother graft 16, which can be done with the aid of rotational angiography, a real time three dimensional road map of the blood vessels B branching off from the aorta A is made and registered in a computer. Using these techniques the position of the side branches can be located quickly and precisely.

Once this road map is complete, a laser L, for example (FIG. 8) can be inserted into the mother graft 16 in order to burn openings O in the wall of the graft 16 to correspond with the computer mapped branching side vessels B (FIG. 8).

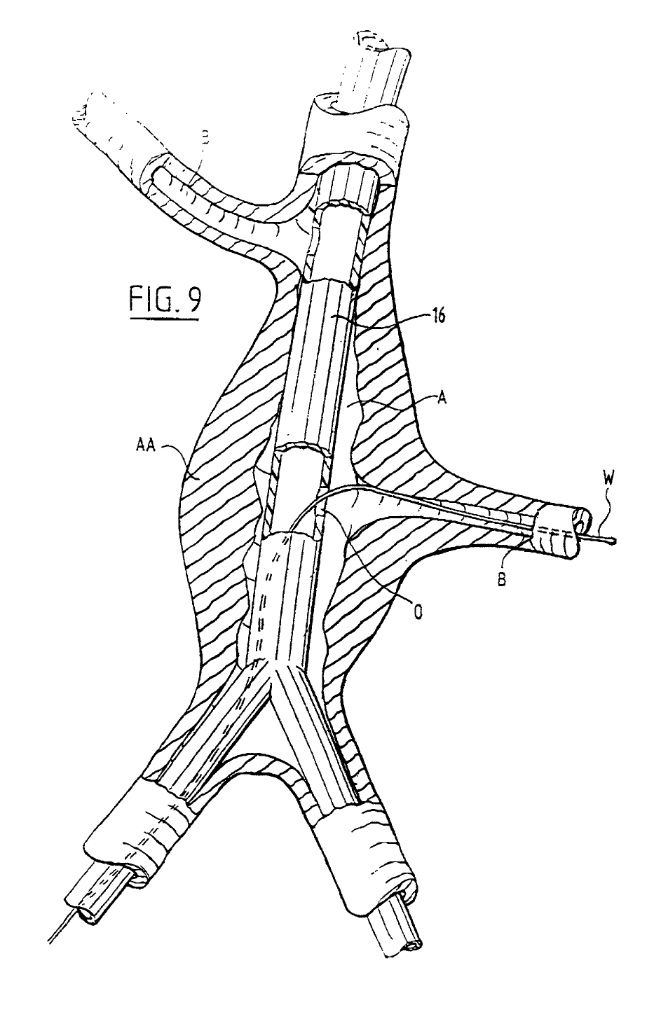

A guidewire W (FIG. 9) is then inserted through the mother graft 16 and through the opening O burned in the side wall thereof and guided through into the branched blood vessels B (FIG. 9).

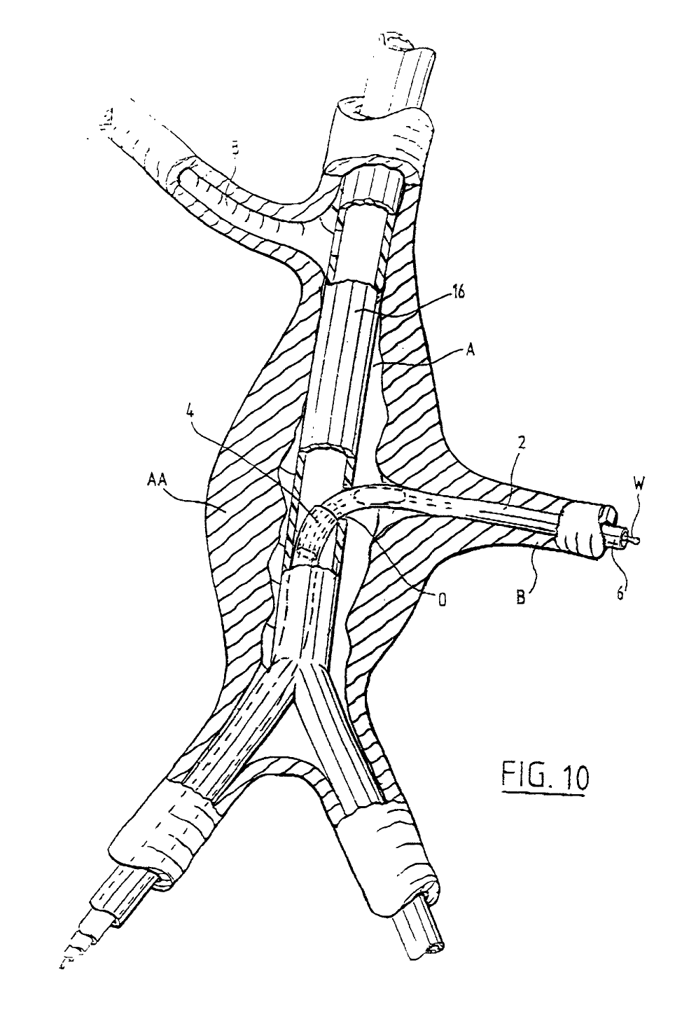

Subsequently, the assembly of the side branch graft 2 in the sheath 4 is inserted over the guide wire W through the mother graft 16, through the openings O, and into the branched blood vessel B, up until the point where an edge of the sheath 4 impinges on the periphery P of the openings O (FIG. 10), At this stage, the graft device 2 is inserted into tee branched blood vessel B and the proximal part 8 thereof remains within the mother graft 16.

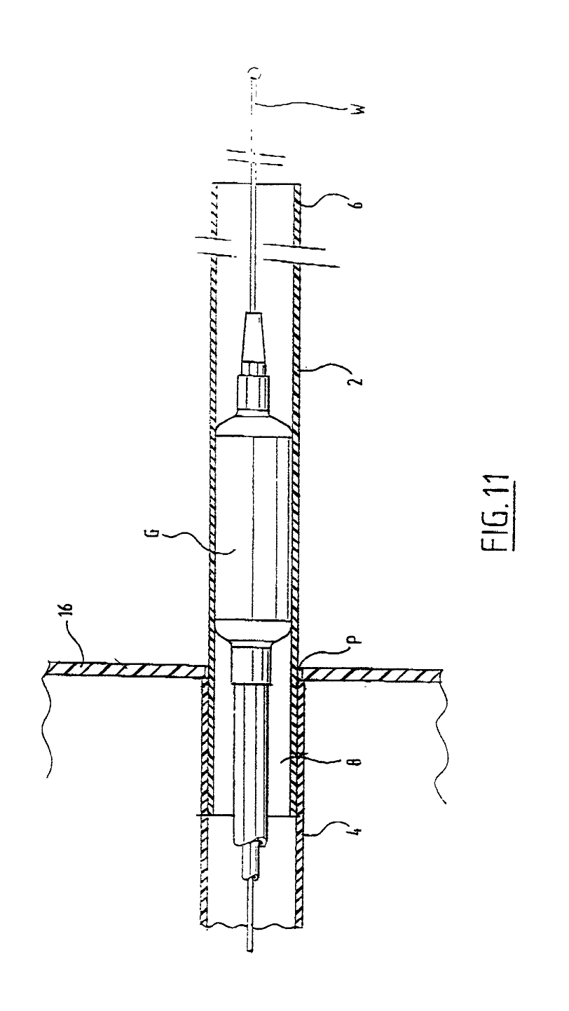

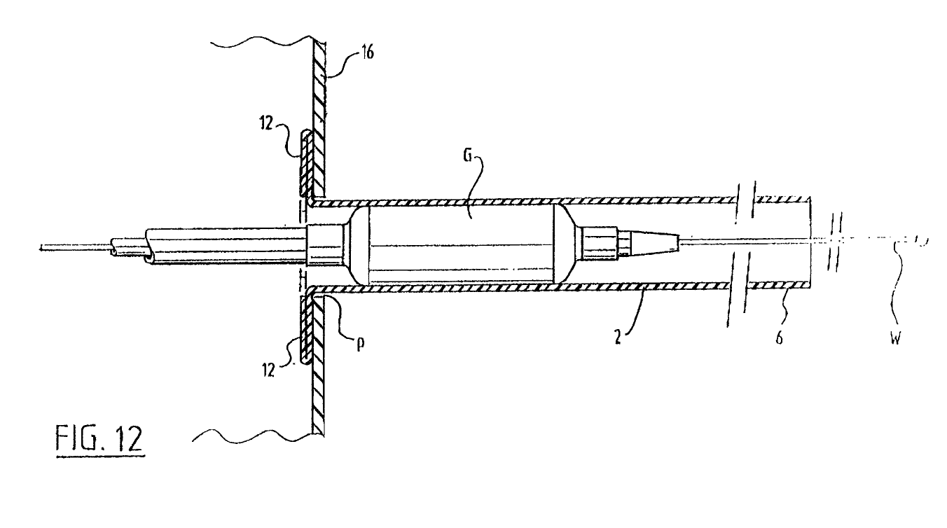

The graft device 2 can be pushed into place by means of a balloon grip G (FIGS. 11 and 12) which is expanded against the interior of the graft device 2 in order to enable this to be arranged into position with a great deal of control.

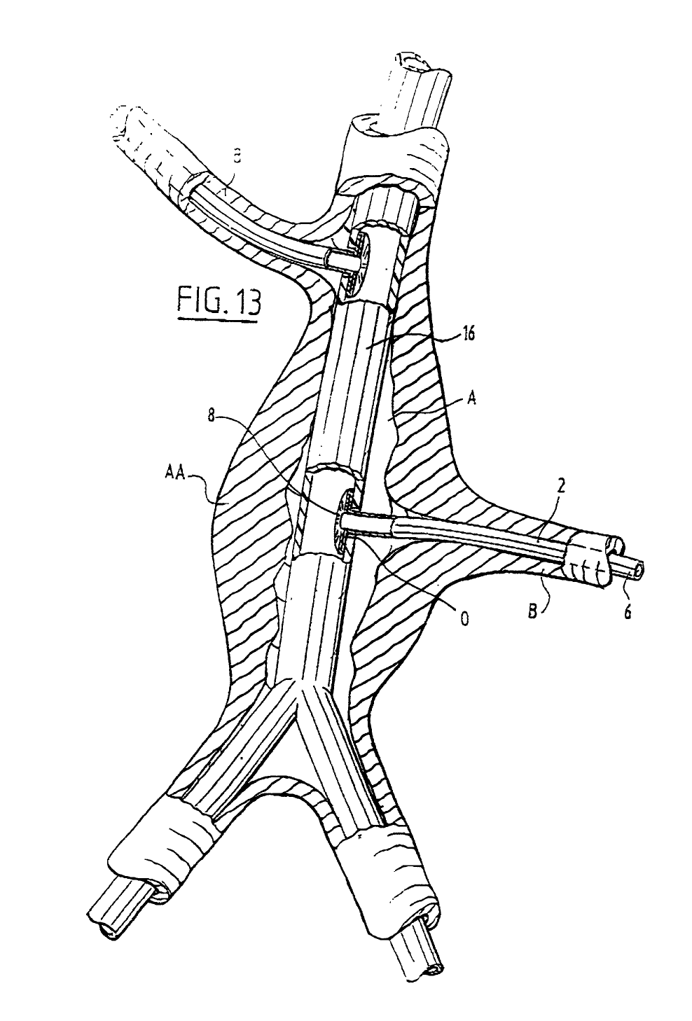

The sheath 4 is then withdrawn whereafter the fingers 12 of the proximal part 8 spring open into the pretensioned rest position (FIGS. 3 to 5) to secure the proximal part 3 of the device 2, within the mother graft 16 (FIGS. 12 and 13). Subsequently, the guidewire W can be removed so that the mother graft 16 has been provided with artificial side branches as it were, in the form of the side branch graft device 2 to ensure blood supply within the branched blood vessels B (FIG. 13).

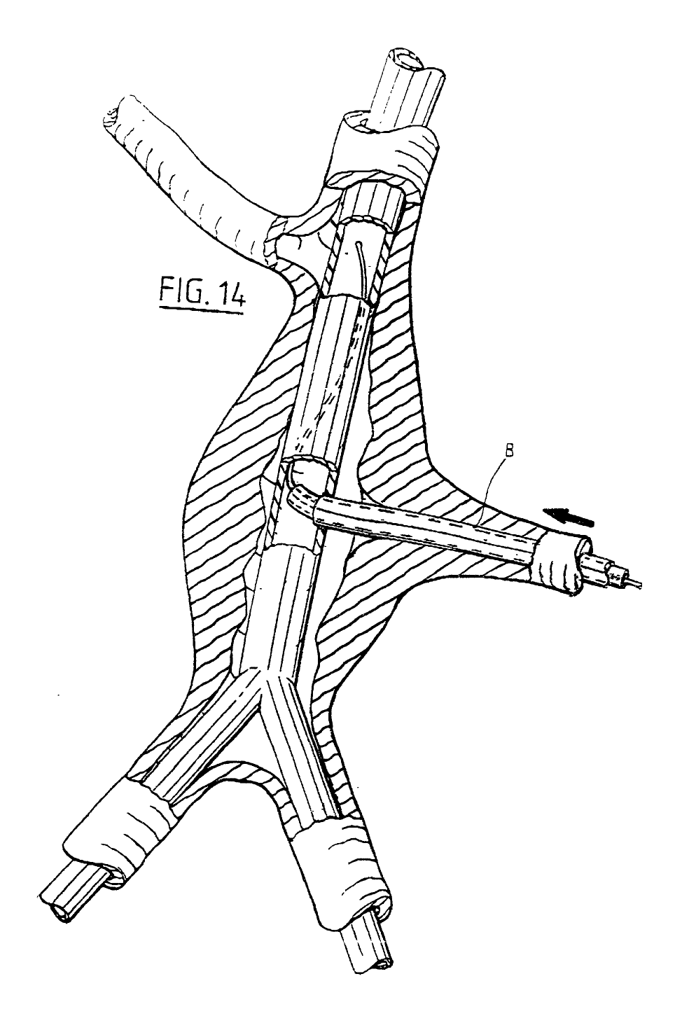

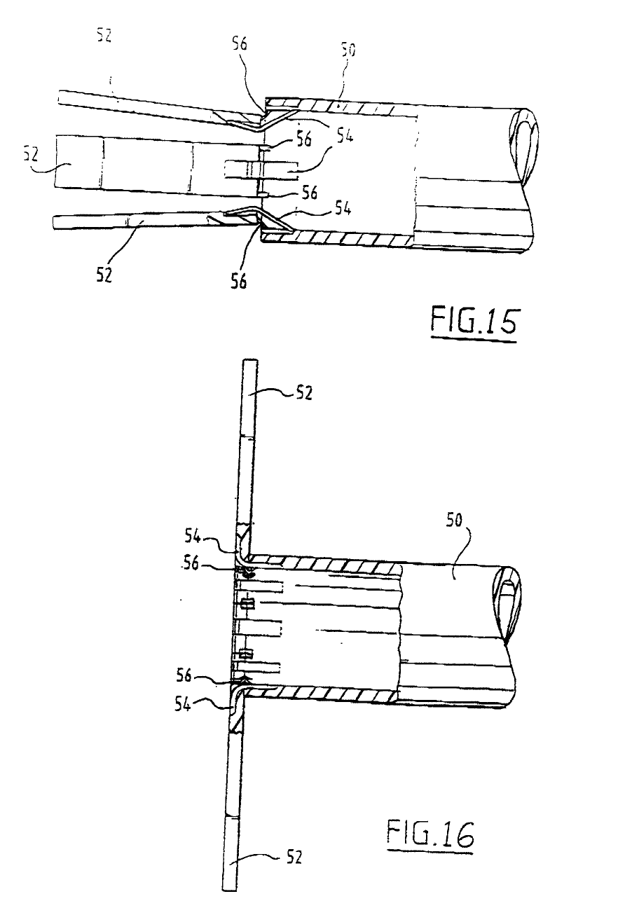

FIG. 14 perspectively shows how the assembly according to the present invention may be inserted, after an open has been arranged in the mother graft, from the outside of the mother graft through a branched blood vessel B.

A further preferred embodiment of the present invention is shown in the partially cut away side view of FIGS. 15 and 16.

In this embodiment, a proximal part of the assembly comprises a stent graft 50 and a plurality of webbed, hinged fingers 52 joined to the stent graft 50 by means of an elbow hinge 54, connected at one end to the stent graft 50 and at the other end thereof to the lower middle finger 52 and on either side thereof by two connecting strips 56, extending from the end of the finger 52 to the opening of the stent graft 50.

The fingers 52 can be clicked outwards, by means for example of an expandable balloon (not shown), so that the fingers 52 click outwards around the elbow hinges 54 and connecting strips 56 to extend at right angles to the stent graft 50. This embodiment provides a so called bi-stable system, whereby the device can be inserted without a covering sheath in a cylindrical form, whereby thereafter the fingers 52 can be clicked open to assume their expanded anchoring position. Accordingly, the fingers of the device according to the present invention may either be self-opening, pre-tensioned, or actively openable, i.e. by means of being clicked between an open and closed position.

Thus, according to the present invention, the device, which functions as a stent for tributaries of large blood vessels, i.e. a stent for side-branches, can be arranged in position either via the side-branch into the main vessel, i.e. from “outside to inside” or via the main vessel into the side-branch, i.e. from “inside to outside”.

The method chosen by the surgeon depends on the accessibility of the treatment site.

Two specific descriptions of arranging the device according to the present invention from “outside to inside” follow:

The first is an alternative for the bifurcated prosthesis, utilised when operating on an aneurysm in the stomach aorta.

According to the present invention a prosthesis can be firstly inserted via the left groin. This prosthesis is tapered whereby the wider end is attached to the aorta and the thinner end attached to the main artery in the groin. Subsequently a guidewire is inserted via the left groin through the artery and transposed therein until it abuts against the prosthesis which has just been arranged in position. An opening is then made in the prosthesis, as described above, and the device according to the present invention is transposed over the guidewire, from outside to inside and placed in the mother prosthesis. This is a very simple and efficient manner of arranging a bifurcated prosthesis.

A second method is the treatment of an aneurysm in the arcus aorta, the artery which first extends upwards from the heart and from where side-branches extend outward to the arms and heart, and which further then bends back on itself to extend down towards the legs.

As a result of arranging this prosthesis, i.e. the mother prosthesis, also called the main catheter, in place in the arcus aorta all the side-branches to the arteries for the arms and neck/head are closed off.

At this point guidewires have already been independently inserted, by the surgeon, into the arteries leading to the arm and the neck arteries, since these are easily accessible via the neck and arms.

After the mother prosthesis has been arranged in position, these guidewires can be transposed through the arm and neck arteries until they abut against the mother prosthesis. In this manner the positioning of the openings in the mother prosthesis is directly determined.

Subsequently the openings are arranged in the mother prosthesis at these abutment points and the guidewires further transposed through these opening whereafter the side-branch stent device according to the present invention can be inserted, from the outside to the inside, over the guidewire.

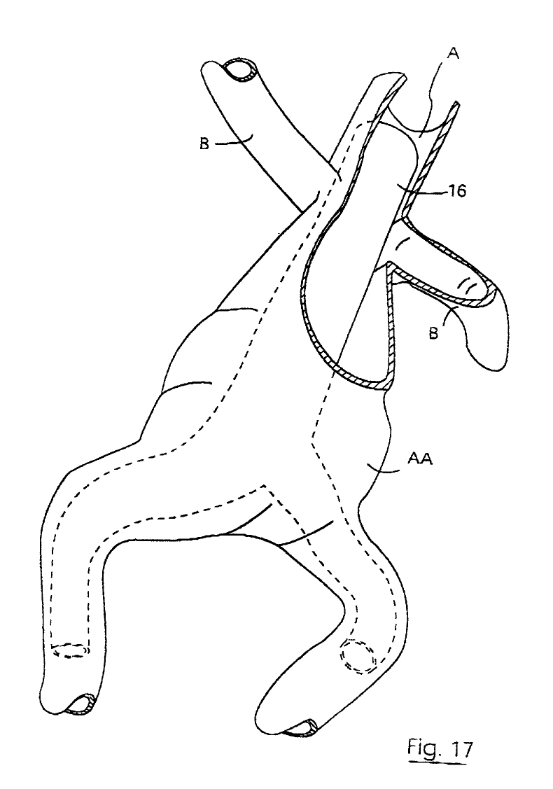

Referring to FIGS. 17 to 22 there is particularly illustrated a technique for grafting side branches from the inside out. In this case the side branches B are renal arteries. However, the same technique may be used to graft other side branch arteries which are not accessible from the outside in, for example, because they terminate in an organ. Such side branches include the mesenteric artery, arteries to the spleen and liver leading from the coeliac trunk, and the lumbar artery. The drawings illustrate grafting of one side branch. The same or a similar technique is also used to provide grafts for other side branches B.

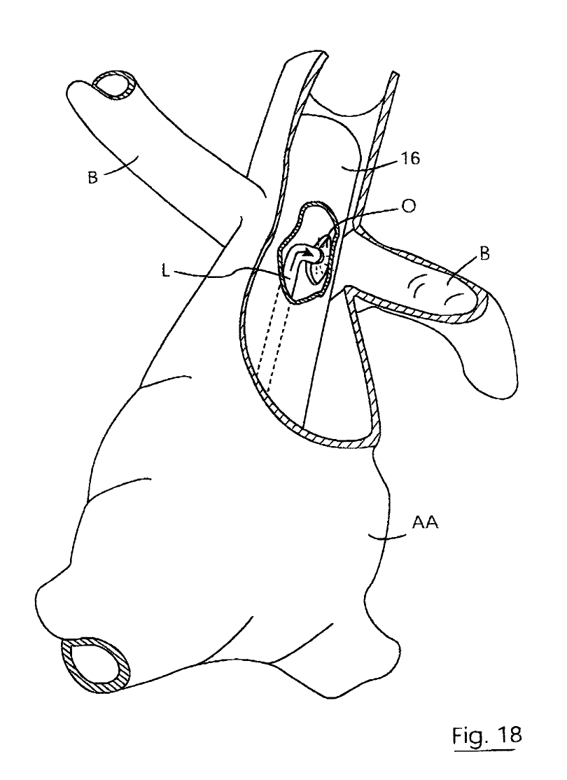

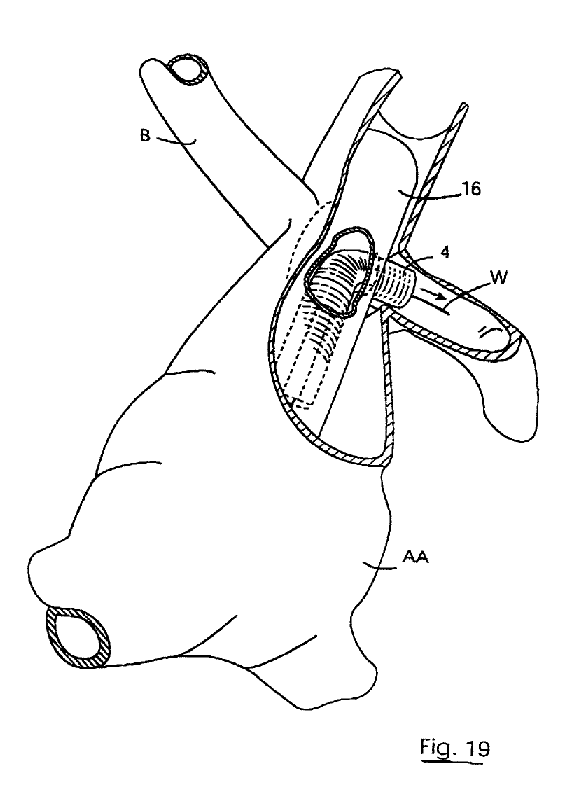

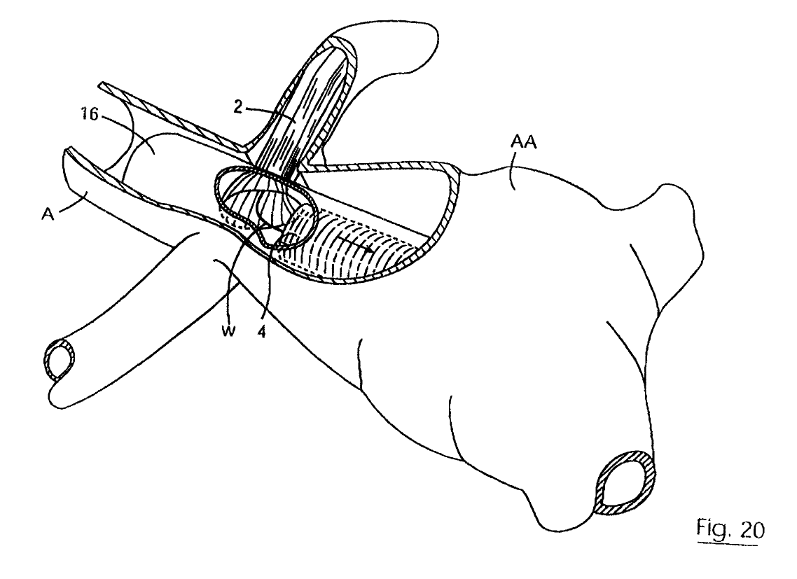

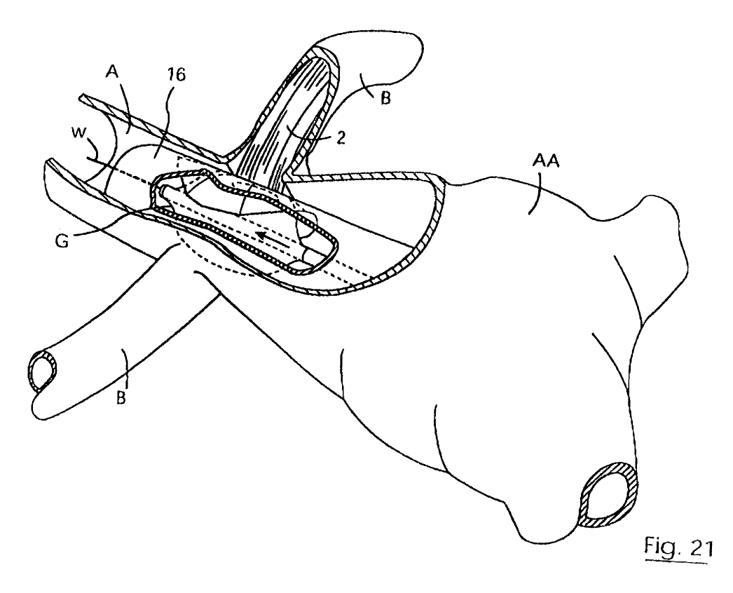

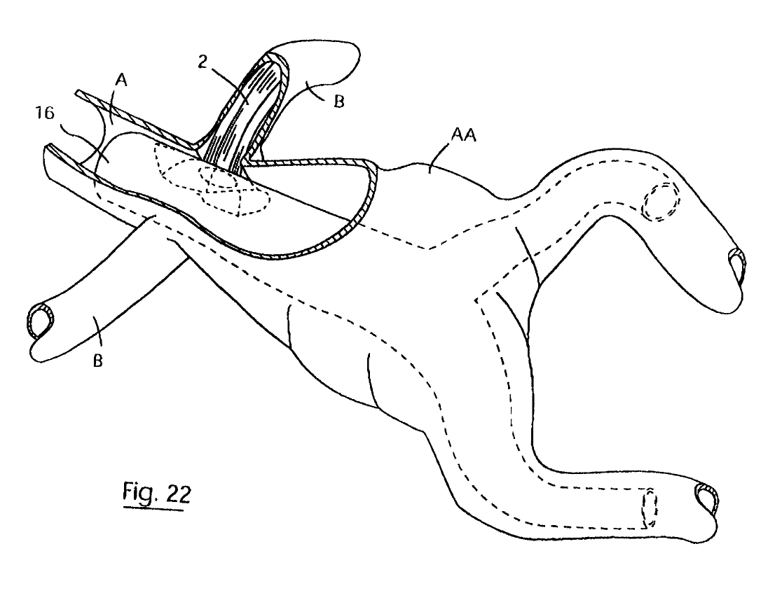

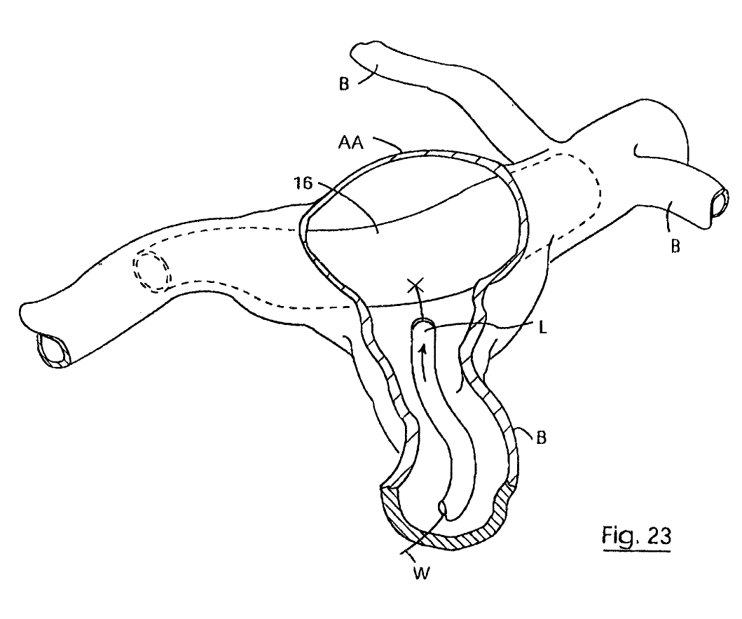

A primary or mother graft 16 is first deployed in the aorta and the position of the side branches which have been closed off by the mother graft 16 are mapped by rotational angiography (FIG. 17). A laser L is tracked over a guidewire through the in other graft 16 and aligned with the entrance to the side branch B. The mother graft 16 is then in situ cut by the laser to provide an opening O from the mother graft 16 into the side branch B (FIG. 18). A guidewire W is tracked into the side branch B and a sheath 4 in which a side graft/device 2 is housed is delivered over the wire into the side branch B (FIG. 19). As described above when the side graft 2 is in position the sheath 4 is withdrawn over the wire W. As the sheath 4 is withdrawn the end of the side branch graft 2 at the opening O is no longer restrained by the sheath 4 and the fingers 12 are free to move from the delivery configuration extending generally parallel to the main body of the side graft 2 to a fixing configuration in which the fingers extend radially outwardly to engage the inner surface of the mother graft 16 in the region of the opening O (FIG. 20). A balloon G may then be introduced through the mother graft 16 and expanded as illustrated in FIG. 21 to assist in fixing the side branch graft 2 in position extending from the mother graft 16. The balloon G and the guidewire W are removed to leave the side branch graft device 2 in position extending from the mother graft 16 (FIG. 22)

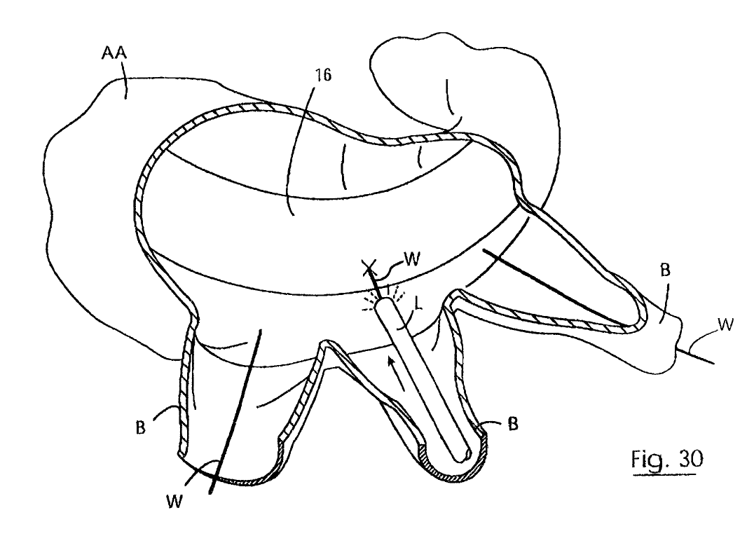

Referring now to FIGS. 23 to 31 there is particularly illustrated a technique for grafting side branches from the outside in. This technique can be used in particular to provide side branch grafts in arteries which are accessible from the outside.

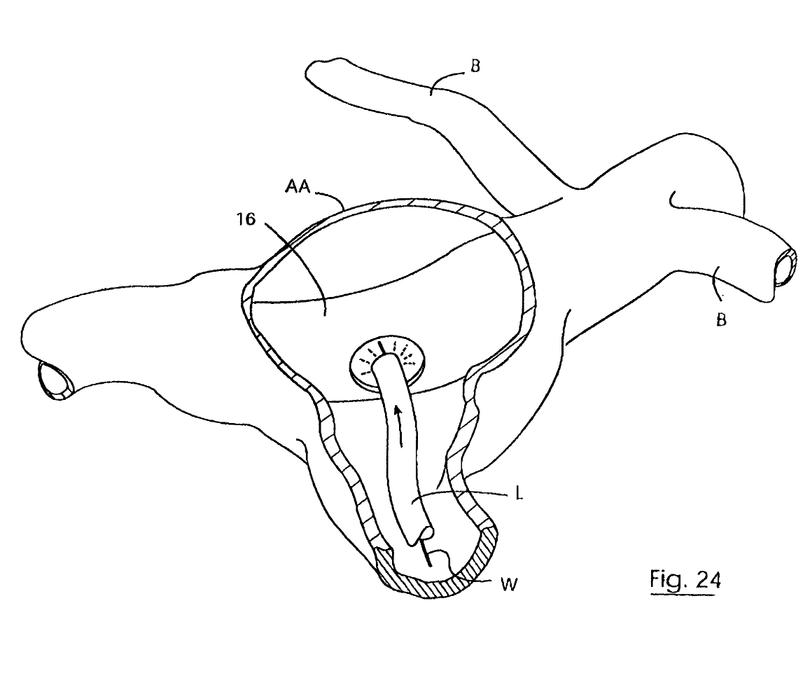

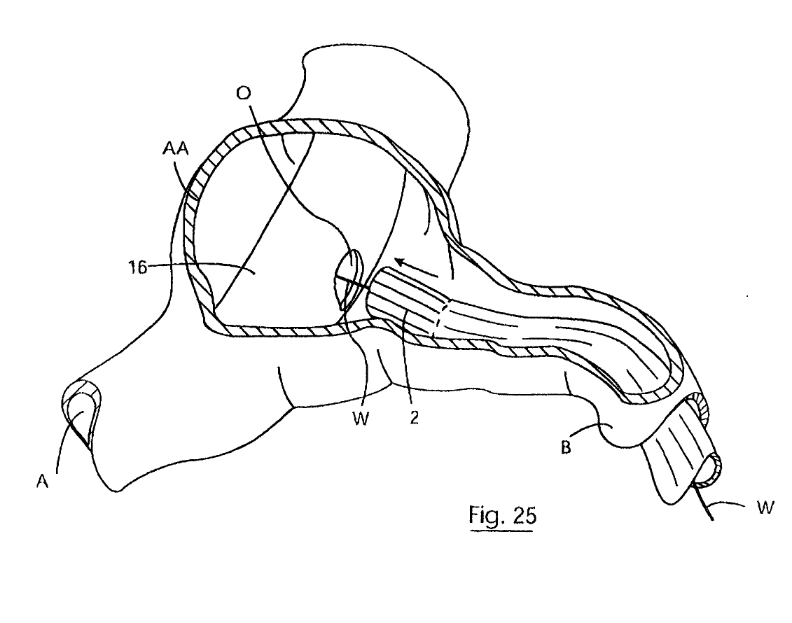

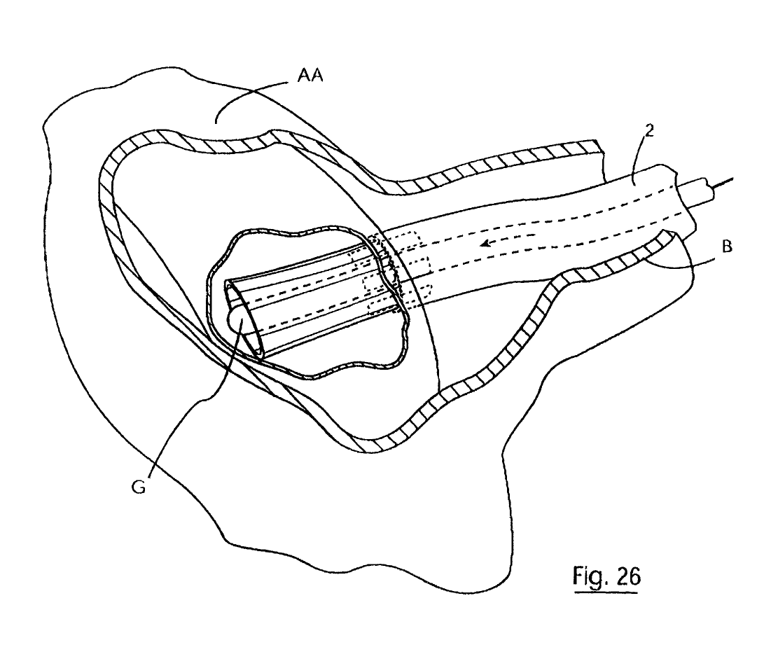

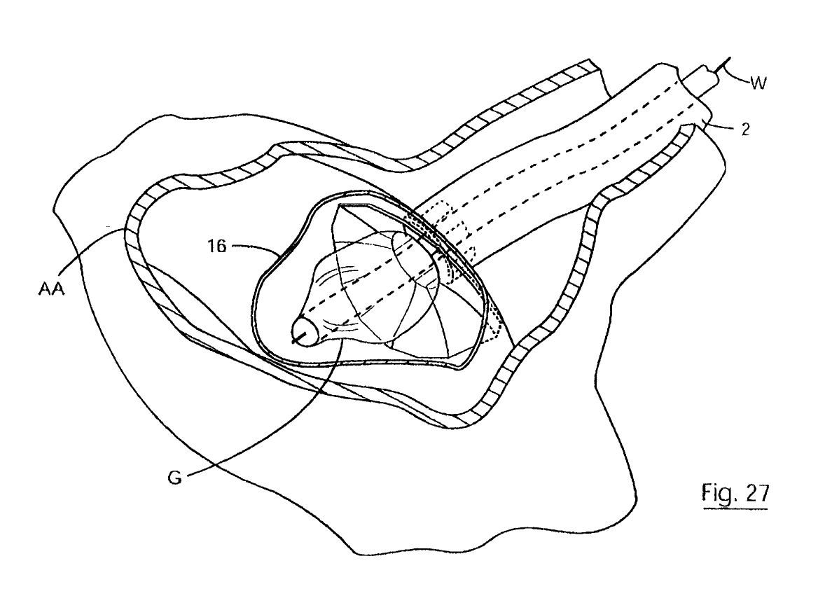

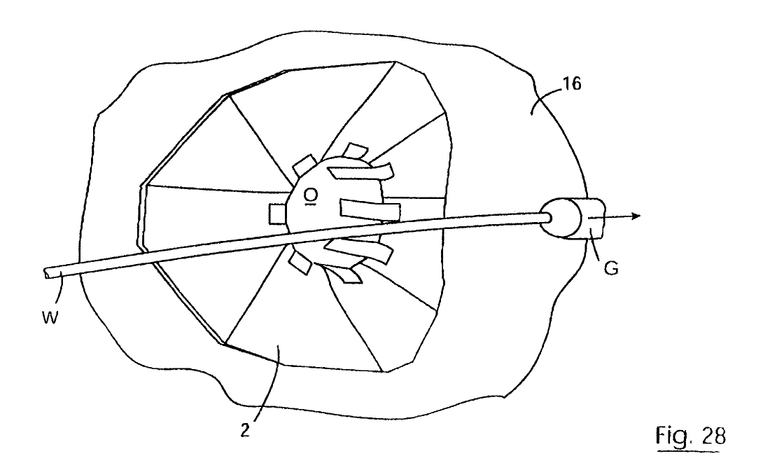

For example, and as particularly illustrated in FIGS. 23 to 28 the technique can be used to provide a graft in an artery with a bifurcation in which a primary or mother graft 16 is first inserted, typically through the right groin and one end is attached to the aorta and the other end attached to the main iliac artery in the groin. A side branch graft 2 of the invention is then deployed in the other leg of the bifurcation. A guidewire W is first inserted via the left groin through the artery until it abuts against the mother graft 16 which has been placed in position. A laser L is then tracked over the guidewire W (FIG. 23) and a hole O is in situ cut in the mother graft 16 (FIG. 24). The laser L is removed and a side branch graft 2 is delivered over the guidewire to the opening O (FIG. 25). The distal end of the graft 2 is then inserted through the opening O (FIG. 26). The distal end of the graft 2 in this case may be of the bistable type described above and may be moved into the deployed configuration by a first balloon G delivered through the side branch (FIG. 27). To further fix the stent graft in position a second balloon may be tracked over a guidewire W through the mother graft 16 to the opening O into the side branch and inflated to press the distal end of the side branch 2 into close engagement with the inner wall of the mother graft 16. The balloon G is then deflated and withdrawn over the guidewire W as illustrated in FIG. 28.

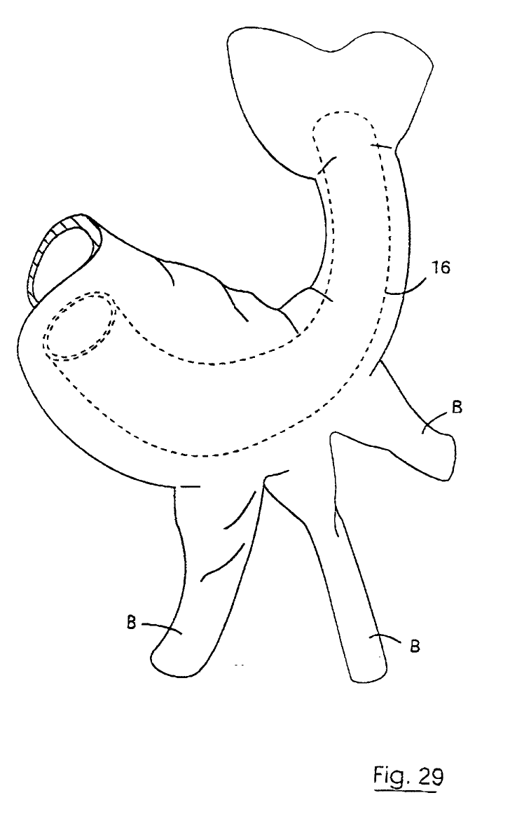

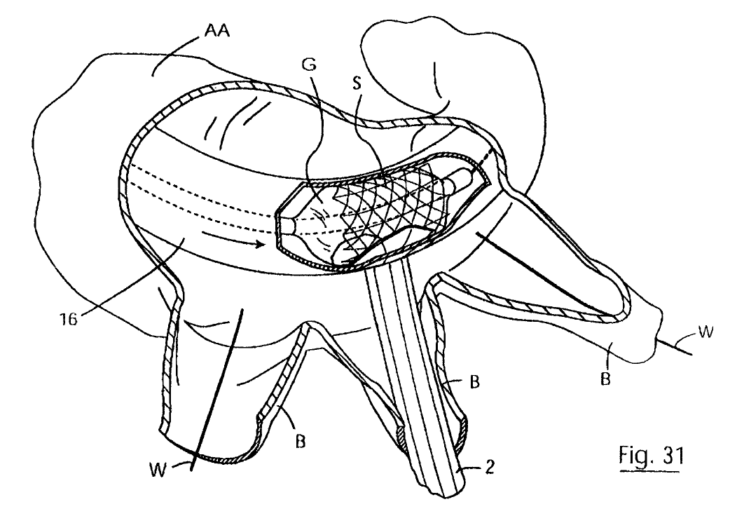

Side branch grafting of the arcus aorta as described above is illustrated in FIGS. 29 to 31 utilising the outside-in technique. In this case a stent S may be deployed by a balloon G at the opening O to the side branch B when the side bran graft 2 is in position. The deployed stents provides a radially outward force on the side branch graft 2 when in position to secure the side branch 2 in close engagement with the inner wail of the mother graft 16, as illustrated in FIG. 31.

The invention is not limited to the embodiments hereinbefore described which may be varied in construction and detail.

1. A method for endoluminal grafting of a body conduit at a site having a side branch comprising the steps of:
introducing a primary graft to a site in the body conduit having a side branch;
mapping the position of the side branch which has been closed off by the primary graft;
providing an opening in the primary graft at the mapped position of the side branch;
introducing a side graft into the side branch; and
fixing the side graft to the primary graft.

2. A method as claimed in claim 1 wherein the position of the side branch is mapped by rotational angiography.

3. A method as claimed in claim 1 wherein the opening in the primary graft is provided by in situ cutting of a side opening in the graft.

4. A method as claimed in claim 3 wherein the primary graft is in situ cut from the inside of the primary graft.

5. A method as claimed in claim 3 herein the primary graft is in situ cut from outside through the side branch.

6. A method as claimed in claim 3 wherein the opening is provided by laser cutting.

7. A method as claimed in claim 1 wherein the side graft is introduced through the side branch.

8. A method as claimed in claim 1 wherein the side graft is introduced through the primary graft.

9. A method as claimed in claim 1 wherein the side graft comprises fixing means for fixing the side graft to the primary graft, the fixing means having a delivery configuration and a stable fixing configurations and the method includes the step of fixing the side graft to the primary graft by moving the fixing means from the delivery configuration to the fixing configuration.

10. A method as claimed in claim 9 wherein the fixing means is moved by an actuator and the method comprises the step of introducing the actuator to the fixing means endoluminally.

11. A method as claimed in claim 10 wherein the actuator comprises an actuating balloon and the method comprises the step of inflating the balloon to move the fixing means from the delivery configuration to the fixing configuration.

12. A method as claimed in claim 9 wherein the method comprises the steps of:
introducing a stabiliser to the fixing means; and
actuating the stabiliser to maintain the fixing means in the fixing configuration.

13. A method for endoluminal grafting of a body conduit at a site having a side branch comprising the steps of:
introducing a graft to a site in the body conduit having a side branch;
mapping the position of the side branch which has been closed off by the primary graft;
in situ cutting an opening in the primary graft at the mapped position of the side branch by introducing a cutter through the primary graft;
introducing a side graft through the primary graft, and into the side branch; and
through the opening in the primary graft fixing the side graft to the primary graft.

14. A method for endoluminal grafting of a body conduit at a site having a side branch comprising the steps of:
introducing a primary graft to a site in the body conduit having a side branch;
mapping the position of the side branch which has been closed off by the primary graft;
in situ cutting an opening in the primary graft at the mapped position of the side branch by introducing a cutter through the side branch;
introducing a side graft through the side branch; and
fixing the side graft to the primary graft.

15. An endoluminal side branch graft comprising a generally tubular body having a proximal part with a proximal opening, a distal part with a distal opening, and a middle section extending between the proximal and distal parts, at least one of the proximal or distal part having fixing means for fixedly positioning the side branch graft to a main branch graft, the fixing means having a delivery configuration and a fixing configuration.

16. A graft as claimed in 15 wherein the delivery configuration is a stable configuration, the fixing configuration is a stable configuration, and the fixing means comprises an integral locking means to lock the fixing means in the stable fixing configuration.

17. A graft as claimed in claim 15 wherein the fixing means comprises a plurality of fingers which extend longitudinally in the delivery configuration and which extend substantially radially outwardly in the fixing configuration.

18. A graft as claimed in claim 17 wherein the fingers are circumferentially spaced-apart in the fixing configuration, preferably a webbing extends between the fingers.

19. A graft as claimed in claim 15 wherein the locking means comprises an elbow hinge.

20. A graft as claimed in claim 17 wherein the locking means comprises an elbow hinge between the fingers and the body of the graft.

21. A graft as claimed in claim 15 comprising anchoring means on the tubular body of the graft preferably the anchoring means comprises one or more hook-like elements for anchoring the graft in position in a side branch.

22. An endoluminal side branch graft system comprising a graft as claimed in claim 15 and a sheath for delivery or retrieval of the graft.

23. A system as claimed in claim 22 wherein the sheath is arrangeable over the side branch graft for introduction and/or removal of the graft from its use site, the sheath when arranged on the side branch graft retaining the fixing means in the delivery configuration.

24. A system as claimed in claim 22 comprising a guide for guiding the assembly to and from the side branch graft use site preferably the guide comprises a guidewire over which the system is guideable.

25. A system as claimed in claim 22 comprising a catheter having an internal diameter substantially greater than the diameter of the side branch graft, whereby the side branch graft is insertable/removable from its use site via an access opening arranged in the mother catheter.

26. A system as claimed in claim 22 comprising a stabiliser to maintain the fixing means in the fixing configuration, preferably the stabiliser is a stent to urge the fixing means into the fixing configuration.

27. An endoluminal graft assembly comprising a graft as claimed in claim 15 and/or a system as claimed in claim 22, and a main branch graft.
